(19) United States
(12) Patent Application Publication
(10) Pub. No.: US 2012/0262672 A1
(43) Pub. Date: Oct. 18, 2012

(54) DOUBLE PASS DEVICE FOR RETINAL IMAGE QUALITY MEASUREMENT

(75) Inventors: Bai-Chuan Jiang, Fort Lauderdale, FL (US); David S. Loshin, Fort Lauderdale, FL (US)

(73) Assignee: NOVA SOUTHEASTERN UNIVERSITY, Fort Lauderdale, FL (US)

(21) Appl. No.: 13/511,519

(22) PCT Filed: Nov. 23, 2010
(86) PCT No.: PCT/US10/57847

§ 371 Date: May 23, 2012

Related U.S. Application Data
(60) Provisional application No. 61/263,990, filed on Nov. 24, 2009.

(51) Int. Cl. A61B 3/10

(52) U.S. Cl. 351/221

(57) ABSTRACT

An optical system for measuring retinal-image quality has a light system constructed and arranged to provide light to be directed onto a retina of a subject's eye, a detection system arranged to receive at least a portion of the light after being reflected or scattered back from the retina of the subject's eye, and an afocal optical system arranged in an optical path of the light between the light system and the detection system. The afocal optical system is constructed and arranged to correct a refractive error of the subject's eye.

### CROSS-REFERENCE OF RELATED APPLICATION

This application claims priority to U.S. Provisional Application No. 61/263,990 filed Nov. 24, 2009, the entire contents of which are hereby incorporated by reference.

### BACKGROUND

1. Field of Invention

The field of the currently claimed embodiments of this invention relate to optical systems, and more particularly to optical systems for measuring retinal-image quality.

2. Discussion of Related Art

Double-pass techniques have been used to objectively measure image quality of the human eye (Santamaria J, Artal P, Bescos J. Determination of the point-spread function of human eyes using a hybrid optical digital method. J Opt Soc Am. A. 1987;4:1109-1114. Artal P, Ferro M, Miranda I, Navarro R. Effects of aging in retinal image quality. J Opt Soc Am. 1993;10:1656-1662.). With this technique, an optical system is used for recording the aerial image of a point source formed by a double-pass through the optical media of the eye. Then, digital image processing procedures are used to determine the point-spread function (PSF) and optical modulation transfer function (MTF) of the eye. However, such systems do not correct for refractive error of the person's eye. Therefore, there remains a need for improved optical systems for measuring retinal-image quality.

### SUMMARY

An optical system for measuring retinal-image quality according to an embodiment of the current invention has a light system constructed and arranged to provide light to be directed onto a retina of a subject's eye, a detection system arranged to receive at least a portion of the light after being reflected or scattered back from the retina of the subject's eye, and an afocal optical system arranged in an optical path of the light between the light system and the detection system. The afocal optical system is constructed and arranged to correct a refractive error of the subject's eye.

### BRIEF DESCRIPTION OF THE DRAWINGS

Further objectives and advantages will become apparent from a consideration of the description, drawings, and examples.

FIG. 1 is a schematic illustration of an optical system for measuring retinal-image quality according an embodiment of the current invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification are incorporated by reference as if each had been individually incorporated.

The term “lens” as used herein is intended to have a broad meaning that can include, but is not limited to, simple thin or thick lenses, compound lens, refractive lenses, diffractive lens, graded refractive index (GRIN) lenses and/or any combination thereof depending on the particular embodiment of the current invention. The term “light” is intended to have a broad meaning to include electromagnetic radiation in both visible regions of the spectrum as well as non-visible regions, such as infrared and near infrared light, for example.

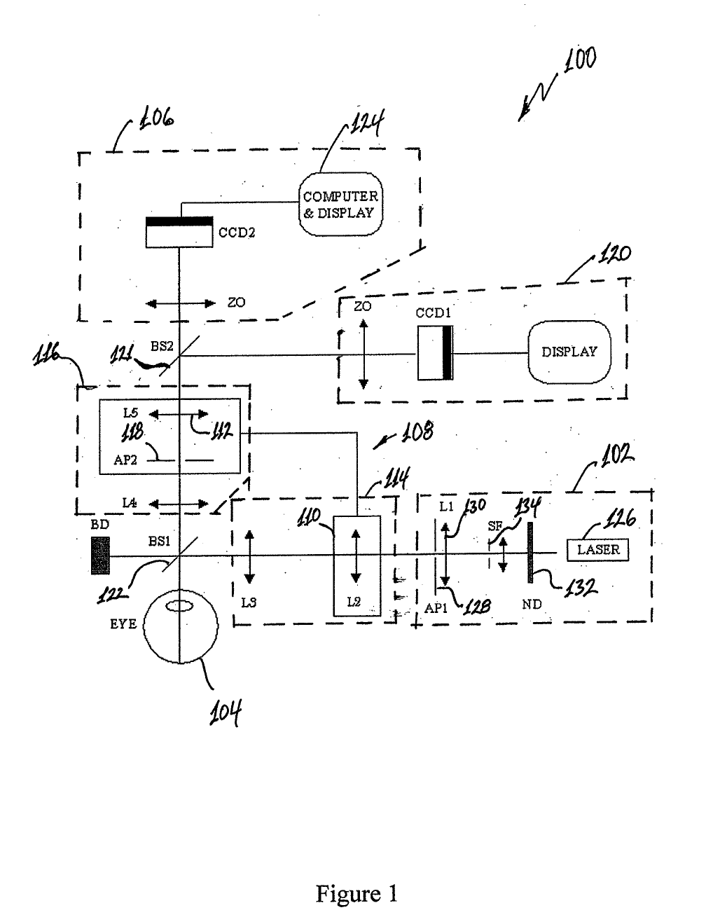

FIG. 1 is a schematic illustration of an optical system 100 for measuring retinal-image quality according to an embodiment of the current invention. The optical system 100 includes a light system 102 constructed and arranged to provide light to be directed onto a retina of a subject's eye 104, a detection system 106 arranged to receive at least a portion of the light after being reflected or scattered back from the retina of the subject's eye 104, and an afocal optical system 108 arranged in an optical path of the light between the light system 102 and the detection system 106. The afocal optical system 108 is constructed and arranged to correct a refractive error of the subject's eye 104.

The afocal optical system 108 includes a first lens 110 that is movable to correct the refractive error of the subject's eye. The first lens 110 can be arranged in an optical path between the light system 102 and the subject's eye 104 in one embodiment of the current invention. Alternatively, the first lens can be arranged in an optical path between the subject's eye 104 and the detection system 106 such as lens 112. In some embodiments of the current invention, the afocal optical system 108 can include a first lens unit 114 arranged in an optical path between the light system 102 and the subject's eye 104, and a second lens unit 116 arranged in an optical path between the subject's eye 104 and the detection system 106 such that at least one of the first lens unit 114 and second lens unit 116 includes a first lens that is movable to correct the refractive error of the subject's eye 104. The first movable lens can be lens 110 and/or 112 in some embodiments of the current invention.

In some embodiments of the current invention, the first lens unit 114 can include the first lens 110 to correct the refractive error of the subject's eye 104 and the second lens unit 116 can include a second lens 112 that is movable to correct the refractive error of the subject's eye 104. In some embodiments, the first lens 110 and the second lens 112 can be coupled to move in a predefined relationship with respect to each other, as is indicated by the solid line in FIG. 1. The first lens unit 114 and second lens unit 116 can also both themselves be afocal lens units in addition to the overall afocal optical system 108 also being afocal.

The second lens unit 116 of the afocal optical system 108 can include an aperture stop 118 arranged to block light scattered from portions of the subject's eye 104 other than the retina. For example, light that is reflected and/or scattered from the cornea or other portions of the eye rather than the retina can obscure the image of light reflected back from the retina. The aperture stop 118 can thus be useful to block the unwanted reflected and scattered light to improve the detection of light reflected and/or scattered from the retina.

In some embodiments of the current invention, the optical system 100 can further include a monitoring system 120 arranged to receive at least a portion of the light after being reflected or scattered back from the retina of the subject's eye 104 such that, for example, alignment can be monitored by a user during operation. For example, a beam splitter 121 can be used to split of some light from the patient's eye, while allowing some light to pass through to the detection system 106. Clearly, the positions of the detection system 106 and the monitoring system could be exchanged in another embodiment of the current invention. The optical system 100 can also include a beam splitter 122 arranged between the first lens unit 114 and the second lens unit 116 of the afocal optical system 108.

The light system 102 can provide a point source of light according to some embodiments of the current invention. The detection system can further include a recording system 124 to record an aerial image of the point source. For example, the detection system can include a computer to record, process and/or display results from measurements with the optical system 100.

In some embodiments of the current invention, the light system 102 can include a high-intensity light source 126. The high-intensity light source can be, but is not limited to, a laser as is indicated in FIG. 1. In some embodiments, an infrared laser, such as a near infrared laser, is suitable. The light system 102 can further include an aperture stop 128 arranged in an optical path from the high-intensity light source 126 at a position that is optically conjugate to an exit pupil of the subject's eye 104. The light system 102 can further include a collimator lens 130 arranged in an optical path from the high-intensity light source 126 between the aperture stop 128 and the high-intensity light source 126. The light system 102 can further include a neutral density filter 132 arranged in an optical path from the high-intensity light source 126 between the high-intensity light source 126 and the collimator lens 130. The light system 102 can further include a spatial filter 134 arranged in an optical path from the high-intensity light source 126 between the neutral density filter 132 and the collimator lens 130

An example of an optical system 100 for measuring retinal-image quality according to an embodiment of the current invention is illustrated schematically in FIG. 1. In that example, the optical system includes four subsystems: (1) a light system including a light source constructed and arranged to provide a beam of light to be directed onto a retina of a subject's eye; (2) an afocal optical system arranged at least partially in an optical path of the light source; (3) a detection system arranged to receive at least a portion of the beam of light after being reflected or scattered back from the retina of the subject's eye; and (4) a monitoring system arranged to receive at least a portion of the beam of light after being reflected or scattered back from the retina of the subject's eye such that alignment can be monitored by a user during operation.

In the embodiment of FIG. 1, the light source provides a uniform and parallel beam of light from a laser, a neutral density filter (ND), a spatial filter (SF), a collimator lens (L₁), and an aperture stop (AP₁). The beam of light, after passing through L₁, becomes parallel and its size is limited by AP₁, which is optically conjugate with the pupil of the eye. The beam is then directed into the main optical path by beam splitter BS₁ to be directed into the eye. An afocal optical system in the embodiment of FIG. 1 includes lenses L₂ and L₃. The afocal optical system is used to control the subject's refractive state (i.e., correct for the subject's refractive error). In this embodiment, the lens L₂ is movable to correct refractive error of the subject's eye.

The detection system can be used to record the aerial image of the point source. The detection system in this embodiment has a CCD camera (CCD2) with a zoom lens system (ZO). A display can also be included. In addition, an afocal system (L₄ and L₅) and aperture stop AP₂ are used to reduce the reflected light from the subject's cornea. In this embodiment, the L₅ and aperture stop AP2 are movable. The movement of the L₅ and AP₂ synchronizes with the movement of the L₂ determined by the subject's refractive error.

A monitoring system can be included to monitor the alignment and record the exit pupil size. The beam splitter BS₂ directs light to a near-infrared CCD camera (CCD1) and a real-time display. In addition, a chin and forehead rest can be included to provide a relatively stable condition for the subject's head and eye positions.

Other variations and substitutions of components of these embodiments are possible without departing from the general concepts of the current invention. Optical systems for measuring retinal-image quality according to some embodiments of the current invention can be useful for fitting contact lenses or intra-ocular lenses, for example. However, the general concepts of the current invention are not limited to only those particular examples of uses.

The embodiments illustrated and discussed in this specification are intended only to teach those skilled in the art the best way known to the inventors to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. An optical system for measuring retinal-image quality, comprising:
a light system constructed and arranged to provide light to be directed onto a retina of a subject's eye;
a detection system arranged to receive at least a portion of said light after being reflected or scattered back from said retina of said subject's eye; and
an afocal optical system arranged in an optical path of said light between said light system and said detection system,
wherein said afocal optical system is constructed and arranged to correct a refractive error of said subject's eye.

2. An optical system for measuring retinal-image quality according to claim 1, wherein said afocal optical system comprises a first lens that is movable to correct said refractive error of said subject's eye.

3. An optical system for measuring retinal-image quality according to claim 2, wherein said first lens is arranged in an optical path between said light system and said subject's eye.

4. An optical system for measuring retinal-image quality according to claim 2, wherein said first lens is arranged in an optical path between said subject's eye and said detection system.

5. An optical system for measuring retinal-image quality according to claim 1, wherein said afocal optical system comprises a first lens unit arranged in an optical path between said light system and said subject's eye, and a second lens unit arranged in an optical path between said subject's eye and said detection system, and wherein at least one of said first and second lens units comprises a first lens that is movable to correct said refractive error of said subject's eye.

6. An optical system for measuring retinal-image quality according to claim 5, wherein said first lens unit comprises said first lens to correct said refractive error of said subject's eye and said second lens unit comprises a second lens that is movable to correct said refractive error of said subject's eye, said first and second lenses being coupled to move in a predefined relationship with respect to each other.

7. An optical system for measuring retinal-image quality according to claim 5, wherein said first lens unit and second lens unit are both afocal lens units.

8. An optical system for measuring retinal-image quality according to claim 5, wherein said second lens unit of said afocal optical system comprises an aperture stop arranged to block light scattered from portions of said subject's eye other than said retina.

9. An optical system for measuring retinal-image quality according to claim 1, further comprising a monitoring system arranged to receive at least a portion of said light after being reflected or scattered back from said retina of said subject's eye such that alignment can be monitored by a user during operation.

10. An optical system for measuring retinal-image quality according to claim 1, wherein said light system provides a point source of light and said detection system further comprises a recording system to record an aerial image of said point source.

11. An optical system for measuring retinal-image quality according to claim 5, further comprising a beam splitter arranged between said first and second lens units of said afocal optical system.

12. An optical system for measuring retinal-image quality according to claim 1, wherein said light system comprises a high-intensity light source.

13. An optical system for measuring retinal-image quality according to claim 12, wherein said light system further comprises an aperture stop arranged in an optical path from said high-intensity light source at a position that is optically conjugate to an exit pupil of said subject's eye.

14. An optical system for measuring retinal-image quality according to claim 13, wherein said light system further comprises a collimator lens arranged in an optical path from said high-intensity light source between said aperture stop and said high-intensity light source.

15. An optical system for measuring retinal-image quality according to claim 14, wherein said light system further comprises a neutral density filter arranged in an optical path from said high-intensity light source between said high-intensity light source and said collimator lens.

16. An optical system for measuring retinal-image quality according to claim 15, wherein said light system further comprises a spatial filter arranged in an optical path from said high-intensity light source between said neutral density filter and said collimator lens.

17. An optical system for measuring retinal-image quality according to claim 12, wherein said high intensity light source is a laser.

18. An optical system for measuring retinal-image quality according to claim 17, wherein said laser is an infrared laser.
